(19) United States
(12) Patent Application Publication
(10) Pub. No.: US 2008/0299668 A1
(43) Pub. Date: Dec. 4, 2008

(54) DETECTION AND QUANTITATION OF CALICHEAMICIN

(75) Inventors: Yuzhu Xue, Somerset, NJ (US); Jennifer Ann Davis, Highland Mills, NY (US); Zhiping Jiang, Nanuet, NY (US); Peter Amorusi, Stony Point, NY (US); Allena Ji Ji, Fair Lawn, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/132,176

(22) Filed: Jun. 3, 2008

Related U.S. Application Data
(60) Provisional application No. 60/933,182, filed on Jun. 4, 2007.

(51) Int. Cl. G01N 33/00

(52) U.S. Cl. 436/94

(57) ABSTRACT

The present invention provides a method of detecting the presence of the calicheamicin component of a calicheamicin-carrier conjugate in a fluid sample, wherein the calicheamicin is covalently bound to the carrier. A bond between the calicheamicin and carrier is disrupted, the calicheamicin portion is released from the calicheamicin-carrier conjugate and detected.

### CROSS-REFERENCE TO RELATED APPLICATIONS

This application claims priority from U.S. Provisional Application No. 60/933,182, filed Jun. 4, 2007, which is incorporated by reference in its entirety.

### FIELD OF THE INVENTION

The present invention is directed to assays for the detection of the calicheamicin portion of a calicheamicin carrier conjugate in general. In particular, the invention is directed to assays wherein the calicheamicin is detected after disruption of a covalent bond between the calicheamicin and the carrier.

### BACKGROUND TO THE INVENTION

Calicheamicin-antibody complexes have been known to be useful in delivering the calicheamicin free radical to a target site for cancer treatment. Such drug-carrier-conjugates have the general structural formula as shown in FIG. 1 and comprise a calicheamicin molecule covalently bound to an antibody. The antibody can be a monoclonal antibody that has specificity for a particular cell type, thus targeting the calicheamicin to the particular cell. In some embodiments, the antibody is bound to calicheamicin via a bifunctional linker that comprises 4-(4-acetylphenoxy)butanoic acid (AcBut) (See, e.g., US Patent Publication 2004/0082764). The bifunctional linker can be bound to the antibody at one end and to calicheamicin by a disulfide bond with a sulfur near the terminus of a methyltruslfide calicheamicin compound. The bifunctional linker between the calicheamicin and the antibody is hydrolysable, allowing for release of the drug from the conjugate after binding to the target. When calicheamicin is covalently bonded to an antibody via a linker in this manner, the calicheamicin component of the molecule is N-acetyl gamma dimethyl hydrazide calicheamicin.

### SUMMARY OF THE INVENTION

It has been discovered that calicheamicin can easily be detected when released from a calicheamicin carrier conjugate. In some embodiments, the release of the calicheamicin involves the disruption of the disulfide bond present between calicheamicin and the bifunctional linker portion of a calicheamicin-antibody conjugate. Once the calicheamicin is separated from the carrier, the detection of the calicheamicin can occur. However, the detection and quantitation of calicheamicin can be hindered by the presence of a 1,4-diylradical generated by the disruption of the disulfide bond within the calicheamicin-carrier conjugate and the molecular rearrangement thereafter. The generation of the calicheamicin free radicals is a direct consequence of reduction of the disulfide bond. The chemical process is outlined as follows:

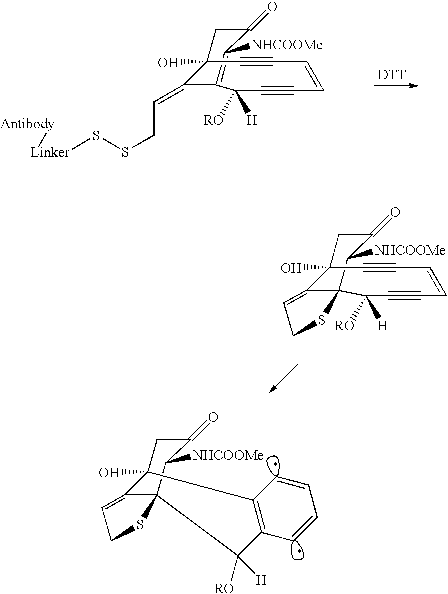

In one embodiment, the invention provides a method for detecting total calicheamicin present in a fluid sample from a subject that has been treated with a drug-carrier conjugate, the method comprising disrupting a bond between the calicheamicin and the carrier and detecting the total amount of calicheamicin present in the sample. In some embodiments, the drug carrier conjugate is calicheamicin bound to an antibody. In some embodiments, the detection of calicheamicin is performed using one or more of the assay techniques of liquid chromatography and mass spectrophotometry. “Total calichemicin” present in the sample refers to the calicheamicin cleaved from the carrier. Calicheamicin that is still bound to the carrier is not detected in the assay of the present invention.

In some embodiments, the disruption of the bond between calicheamicin and the carrier comprises the disruption of a disulfide bond between a bifunctional linker that links the calicheamicin to the carrier. In some embodiments, the disulfide bond is disrupted by the addition of a reducing agent. In some embodiments, the present invention further provides the use of a free radical scavenger to terminate the free radical chain reaction generated by disruption of the disulfide bond linking calicheamicin to the bifunctional linker.

In one embodiment, the invention provides a method to detect total calicheamicin in a sample after dissociation of a calicheamicin-monoclonal antibody conjugate, the method comprising the following steps: a) disrupting a covalent bond between calicheamicin and the monoclonal antibody and b) detecting the calicheamicin released from the calicheamicin-antibody conjugate. In some embodiments, the covalent bond disrupted between calicheamicin and the antibody component is a disulfide bond between the bifunctional linker linking the antibody to the calicheamicin and the calicheamicin. In some embodiments, the disulfide bond is disrupted by the addition of a reducing agent.

In some embodiments, the invention provides a method to detect total calicheamicin after dissociation of the calicheamicin-monoclonal antibody complex, the method comprising the following steps: a) disrupting the disulfide covalent bond between calicheamicin and the carrier component and b) detecting the calicheamicin released from the calicheamicin-carrier conjugate.

In other embodiments, the invention provides a method to detect total calicheamicin after dissociation of the calicheamicin-monoclonal antibody complex, the method comprising the following steps: a) disrupting a covalent bond between calicheamicin and a linker which links the calicheamicin to the carrier component wherein the covalent bond is disrupted by hydrolysis of the bifunctional linker linking calicheamicin to the antibody and b) detecting the calicheamicin released from the calicheamicin-carrier conjugate. The hydrolysis occurs on the bond of C═N and the hydrolysis product is called NAc-gamma-calicheamicin DMH.

In one embodiment, the invention provides any one or more of the methods described herein wherein the method used to detect the calicheamicin released from the drug-carrier conjugate includes mass spectrometry. In another embodiment, the invention provides any one or more of the methods described herein wherein the method used to detect the calicheamicin released from the drug-carrier conjugate is a process selected from the group consisting of: Surface Enhanced Laser Desorption Ionization (SELDI); Matrix-Assisted Laser Desorption/Ionization quadropole time-of-flight (MALDI-TOF); multiple sequential mass spectrometry (MS/MS), sequential time-of-flight (TOF-TOF), electrospray ionization quadropole time-of-flight (ESI-O-TOF) and ION-TRAP. In some embodiments, the mass spectrometry used to detect calicheamicin is multiple sequential spectrometry and comprises liquid chromatography LC/MS/MS.

In another embodiment, the invention provides any one or more of the methods described herein wherein the disruption of the disulfide bond present in the calicheamicin-carrier conjugate is disrupted by the addition of a reducing agent. In some embodiments the reducing agent is a thiol reducing agent. In some preferred embodiments the method for disrupting the disulfide bond present in the calicheamicin-carrier conjugate is carried out by adding dithiothreitol (DTT).

In another embodiment, the invention provides any one or more of the methods described herein wherein the disruption of the covalent bond between calicheamicin and the carrier component of the drug-carrier conjugate is disrupted by hydrolysis.

In some embodiments the method for disrupting the covalent bond between calicheamicin and the carrier comprises incubating the sample in an acidic pH. In some embodiments, the acidic pH is between about 2.0 and about 4.0. In some embodiments, the pH. Is between 3.0 and 4.0 or any ranges or intervals between these pH levels.

In some embodiments, the invention provides any one or more of the methods described herein wherein a free radical scavenger is added to the sample, wherein the free radical scavenger is selected from the group consisting of: an alcohol, a thiol derivative, Tris(2-Carboxyethyl)phosphine; benzoic acid; a carbonate ion; a metal complex such as copper complex, manganese complex; sodium hydrogensulfite; sodium sulfite; sodium metabisulfite; nordihydroguaiaretic acid; propyl gallate; butylhydroxyanisole, dibutylhydroxytoluene; erythorbic acid; sodium erythorbate, ascorbyl palmitate, ascorbyl dipalmitate; ascorbyl stearate; sodium ascorbate; calcium ascorbate; glutathione; and uric acid. In a more preferred embodiment the free radical scavenger is an alcohol comprising isopropyl alcohol.

In another embodiment, the invention provides any one or more of the methods described herein wherein the carrier conjugated to the calicheamicin is selected from the group consisting of: mono- and polyclonal antibodies and their chemically or genetically manipulated counterparts; their antigen-recognizing fragments and their chemically or genetically manipulated counterparts; small modular immunopharmaceuticals (SMIPs) and their chemically or genetically manipulated counterparts; nanobodies and their chemically or genetically manipulated counterparts; soluble receptors and their chemically or genetically manipulated counterparts; growth factors and their chemically or genetically manipulated counterparts; aptamers; liposomes; non-glycosylated proteins; and nanoparticles. In some embodiments, the carrier is one that can specifically bind to an antigen expressed on or within cancer cells. In some embodiments the antigen expressed on or within the cancer cells is selected from the group consisting of: 5T4; CD19; CD20; CD22; CD33; CD40; EphA2; Lewis Y; HER-2; type I Fc receptor for immunoglobulin G (Fc gamma R1); CD52; epidermal growth factor receptor (EGFR); vascular endothelial growth factor (VEGF); DNA/histone complex; carcinoembryonic antigen (CEA); CD47; CD105 (endoglin); folate R: CSPG4 (melanoma-associated antigen); MUC-1; prostate specific membrane antigen (PSMA); VEGFR2 (vascular endothelial growth factor receptor 2 or kinase insert domain-containing receptor, KDR); epithelial cell adhesion molecule (Ep-CAM); fibroblast activation protein (FAP); Trail receptor-1 (DR4); progesterone receptor; oncofetal antigen CA 19.9; and fibrin.

In another embodiment, the invention provides one or more of the methods described herein wherein the carrier is a monoclonal antibody. In some embodiments, the monoclonal antibody is selected from the group consisting of: an anti-CD22 monoclonal antibody; an anti-5T4 monoclonal antibody; anti-CD33 antibody; and an anti-Lewis Y monoclonal antibody.

In another embodiment, the invention provides one or more of the methods described herein wherein the calicheamicin is N-acetyl gamma dimethyl hydrazide calicheamicin.

In another embodiment, the invention provides one or more of the methods described herein wherein the N-acetyl gamma dimethyl hydrazide calicheamicin is conjugated to a carrier monoclonal antibody and the monoclonal antibody is one that can specifically bind to an antigen selected from the group consisting of 5T4; CD19; CD20; CD22; CD33; CD40; EphA2; Lewis Y; HER-2; type I Fc receptor for immunoglobulin G (Fc gamma R1); CD52; epidermal growth factor receptor (EGFR); vascular endothelial growth factor (VEGF); DNA/histone complex; carcinoembryonic antigen (CEA); CD47; CD105 (endoglin); folate R; CSPG4 (melanoma-associated antigen); MUC-1; prostate specific membrane antigen (PSMA); VEGFR2 (vascular endothelial growth factor receptor 2 or kinase insert domain-containing receptor, KDR); epithelial cell adhesion molecule (Ep-CAM); fibroblast activation protein (FAP); Trail receptor-1 (DR4); progesterone receptor; oncofetal antigen CA 19.9; and fibrin.

In another embodiment, the invention provides one of the methods described herein wherein the calicheamicin-carrier conjugate undergoes disulfide bond disruption to form N-acetyl gamma dimethyl hydrazide calicheamicin and the carrier. In some embodiments, the N-acetyl gamma dimethyl hydrazide calicheamicin undergoes a molecular rearrangement to form N-acetyl epsilon calicheamicin and is detected by mass spectrometry.

In another embodiment, the invention provides any one or more of the methods described herein wherein the disruption step to disrupt the disulfide bond in the calicheamicin-carrier conjugate is followed by an incubation step for a time period between about 24 hours and 96 hours prior to detecting the calicheamicin portion released from the conjugate. In one embodiment, the disruption step is followed by an incubation step for a time period between about 36 hours and 72 hours. In one embodiment, the disruption step is followed by an incubation step for about 48 hours prior detecting the released calicheamicin.

In one embodiment, the invention provides any one or more of the methods described herein wherein the disruption step to disrupt the disulfide bond in the calicheamicin-carrier conjugate is incubated at a temperature between about −20° C. and 37° C. In some embodiments, the disruption step is followed by an incubation step at a temperature between about 0° C. and 20° C. prior to detecting the calicheamicin portion released from the conjugate. In one embodiment the incubation step is incubated at a temperature of 4° C.

In one embodiment, the invention provides a kit for determining the concentration of calicheamicin present in a sample comprising: instructions for disrupting a disulfide bond between calicheamicin and an antibody; a free radical scavenger; and a reducing agent.

The assays described herein can be used, e.g., to quantitate the amount of total calicheamicin present in a subject's serum following administration of a calicheamicin-antibody conjugate.

### BRIEF DESCRIPTION OF THE DRAWINGS

FIG. 1 shows a diagrammatic and chemical structural representation of a calicheamicin-antibody drug-carrier conjugate.

FIG. 2 shows a graphical representation of a calibration curve generated by mass spectrophotometry analysis of varying concentrations of calicheamicin.

FIG. 3A shows a graphical representation of mass spectrophotometry analysis of N-acetyl epislon calicheamicin in control rat serum spiked with the internal standard.

FIG. 3B shows a graphical representation of mass spectrophotometry analysis of N-gamma calicheamicin from control rat serum spiked with the internal standard. Epsilon calicheamicin is formed from the N-gamma calicheamicin.

FIG. 4A shows a graphical representation of mass spectrophotometry analysis of N-acetyl epislon calicheamicin from a CME-548 LLOQ Calibration Standard (2.00 ng Calicheamicin/mL) in Rat Serum.

FIG. 4B shows a graphical representation of mass spectrophotometry analysis of N-gamma calicheamicin from a CME-548 LLOQ Calibration Standard (2.00 ng Calicheamicin/mL) in Rat Serum. Epsilon calicheamicin is formed from the N-gamma calicheamicin.

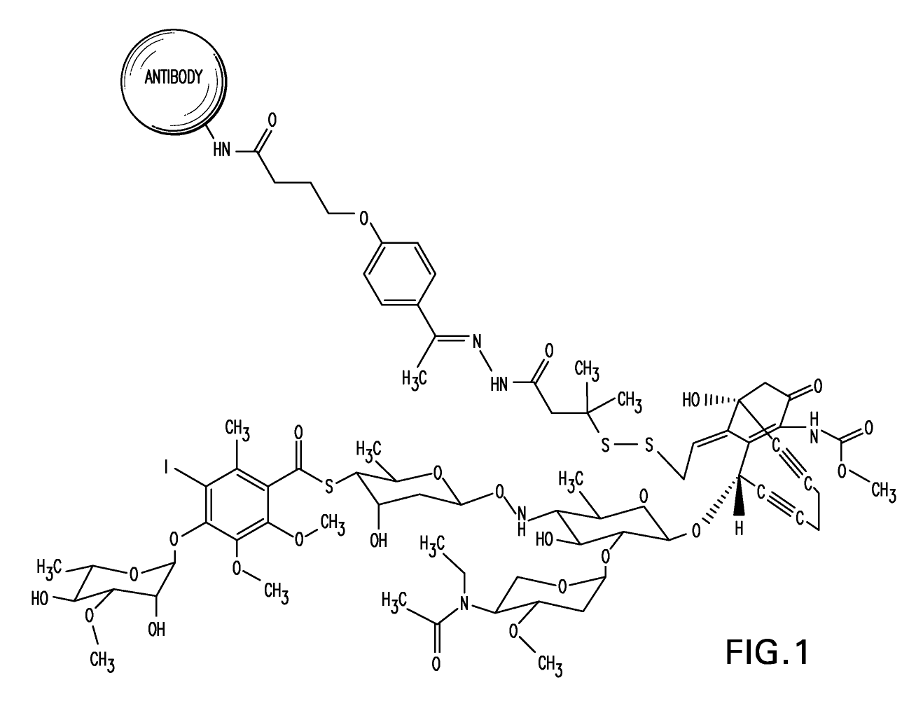

### DETAILED DESCRIPTION OF THE INVENTION

Drug conjugates developed for systemic pharmacotherapy are target-specific cytotoxic agents. The concept involves coupling a therapeutic agent to a carrier molecule with specificity for a defined target cell population. As used herein, such drug conjugates are also referred to as “drug-carrier conjugates.”

Antibodies with high affinity for antigens are a natural choice as targeting moieties or as the “carrier” portion of a drug-carrier conjugate. With the availability of high affinity monoclonal antibodies, the prospects of antibody-targeting therapeutics have become promising. Toxic substances that have been conjugated to monoclonal antibodies include toxins, low-molecular-weight cytotoxic drugs, biological response modifiers, and radionuclides. Antibody-toxin conjugates are frequently termed immunotoxins, whereas immunoconjugates consisting of antibodies and low-molecular-weight drugs such as methothrexate and Adriamycin are called chemoimmunoconjugates. Immunomodulators contain biological response modifiers that are known to have regulatory functions such as lymphokines, growth factors, and complement-activating cobra venom factor (CVF). Radioimmunoconjugates consist of radioactive isotopes, which may be used as therapeutics to kill cells by their radiation or used for imaging. Antibody-mediated specific delivery of cytotoxic drugs to tumor cells is expected to not only augment their anti-tumor efficacy, but also prevent nontargeted uptake by normal tissues, thus increasing their therapeutic indices.

A number of antibody-based therapeutics for treating a variety of diseases including cancer and rheumatoid arthritis have been approved for clinical use or are in clinical trials for a variety of malignancies including B-cell malignancies such as Non-Hodgkin's lymphoma. One such antibody-based therapeutic is rituximab (Rituxan™), an unlabelled chimeric human (1 (+m(1V-region) antibody, which is specific for cell surface antigen CD20, which is expressed on B-cells. These antibody based therapeutics rely either on complement-mediated cytotoxicity (CDCC) or antibody-dependent cellular cytotoxicity (ADCC) against B cells, or on the use of radionuclides, such as ¹³I or ⁹⁰Y, which have associated preparation and use problems for clinicians and patients. Consequently, there is a need for the generation of immunoconjugates that can overcome the shortcomings of current antibody-based therapeutics to treat a variety of malignancies including hematopoietic malignancies like non-Hodgkin's lymphoma (NHL), which can be produced easily and efficiently, and which can be used repeatedly without inducing an immune response.

The development of specific binding assay techniques has provided extremely useful analytical methods for determining various substances of diagnostic, medical, environmental and industrial importance that appear in various liquid or solid (e.g., tissue) samples at very low concentrations. Specific binding assays are based on the specific interaction between a bindable analyte under determination and a binding partner therefore, i.e. analyte-specific moiety. The binding of the analyte-specific moiety and interaction of any additional reagents, if necessary, effect a mechanical separation of bound and unbound labeled analyte or affect the label in such a way as to modulate the detectable signal. The former situation is normally referred to as heterogeneous and the latter as homogeneous, in that the latter does not require a separation step.

Immunoconjugates comprising a member of the potent family of antibacterial and antitumor agents, known collectively as the calicheamicins or the LL-E33288 complex, (see U.S. Pat. No. 4,970,198 (1990)), were developed for use in the treatment of myelomas. The most potent of the calicheamicins is designated (1, which is herein referenced simply as gamma. These compounds contain a methyltrisulfide that can be reacted with appropriate thiols to form disulfides, at the same time introducing a functional group such as a hydrazide or other functional group that is useful in attaching a calicheamicin derivative to a carrier. (See U.S. Pat. No. 5,053,394). MYLOTARG® (Sievers, E. L. et al (1999) Blood: 93, 3678-3684), also referred to as CMA-676 or CMA, is a commercially available drug that comprises calicheamicin bound to a monoclonal antibody as carrier. MYLOTARG® (gemtuzumab ozogamicin) is currently approved for the treatment of acute myeloid leukemia in elderly patients. The drug consists of an antibody against CD33 that is bound to calicheamicin by means of an acid-hydrolyzable linker. The disulfide analog of the semi-synthetic N-acetyl gamma calicheamicin was used for conjugation (U.S. Pat. Nos. 5,606,040 and 5,770,710).

### DEFINITIONS

As used in the specification and the appended claims, the singular forms “a,” “an” and “the” include reference to the plural unless the context clearly dictates otherwise. Thus, for example, reference to “an antibody” includes a plurality of such compositions, i.e., “antibodies.”

The term “about” means within a statistically meaningful range of a value. Such a range can be within an order of magnitude, typically within 20%, more preferably within 10%, and more preferably within 5% of a stated measurement or concentration. The allowable variation encompassed by the term “about” depends on the particular system under study, and can be readily appreciated by one of ordinary skill in the art.

Unless the context specifically indicates otherwise, the term “antibody” as used herein is meant to include one or more of mono- and polyclonal antibodies and their chemically or genetically manipulated counterparts; their antigen-recognizing fragments and their chemically or genetically manipulated counterparts; and synthetic molecules comprising their antigen-recognizing fragments.

As used herein, the term “carrier or carrier molecule” includes, but is not limited to: mono- and polyclonal antibodies and their chemically or genetically manipulated counterparts; their antigen-recognizing fragments and their chemically or genetically manipulated counterparts; small modular immunopharmaceuticals (SMIPs) and their chemically or genetically manipulated counterparts; nanobodies and their chemically or genetically manipulated counterparts; soluble receptors and their chemically or genetically manipulated counterparts; growth factors and their chemically or genetically manipulated counterparts; aptamers; liposomes; non-glycosylated proteins; and nanoparticles; aptamers; liposomes; non-glycosylated proteins; and nanoparticles.

A drug “functionalized” or “derivatized” to enable conjugation with an antibody as used herein is also meant to include the “drug” portion of a “drug-carrier conjugate”. Examples of the antibodies, as part of the drug-carrier conjugate, are antibodies specific for CD22, 5T4, CD33 and Lewis-Y antigens. The antibodies each are specific for a different receptor and specifically bind to an antigen expressed on cancer cells.

“Disruption” of the calicheamicin-carrier conjugate to generate a “carrier portion” and a “calicheamicin portion” results in separation of the calicheamicin from the antibody. “Total calicheamicin” is calicheamicin that is cleaved from the carrier due to the disruption of the calicheamicin-carrier conjugate. Any calicheamicin remaining conjugated to the carrier is not measured in the assay of the present invention.

In particular embodiments of the invention, the drug portion comprises a cytotoxic agent such as a calicheamicin, also called the LL-E33288 complex, for example, gamma-calicheamicin_{γ1}. See U.S. Pat. No. 4,970,198. Early studies with antibody conjugates of gamma calicheamicin hydrazide derivatives showed antigen-based cytotoxicity in vitro and activity in xenograft experiments. Stabilizing the disulfide bond that is present in all calicheamicin conjugates by adding dimethyl substituents made additional improvements.

Additional examples of calicheamicins suitable for use in preparing antibody/drug conjugates of the invention are disclosed in U.S. Pat. Nos. 4,671,958; 5,053,394; 5,037,651; 5,079,233; and 5,108,912; which are incorporated herein in their entirety. These compounds contain a methyltrisulfide that may be reacted with appropriate thiols to form disulfides, at the same time introducing a functional group such as a hydrazide or other functional group that is useful for conjugating calicheamicin to an antibody. Stabilizing the disulfide bond that is present in all calicheamicin conjugates by adding dimethyl substituents made additional improvements. This led to the choice of N-acetyl gamma calicheamicin dimethyl hydrazide, or NAc-gamma DMH, as one of the optimized derivatives for conjugation. Disulfide analogs of calicheamicin can also be used, for example, analogs described in U.S. Pat. Nos. 5,606,040 and 5,770,710, which are incorporated herein in their entirety.

Representative methods for preparing antibody-drug conjugates include those described in U.S. Pat. No. 5,053,394, U.S. patent application Publication No. 2004-0082764A1 and U.S. patent application Publication No. 2004-0192900. Conjugation may be performed using the following conditions: 10 mg/ml antibody, 8.5% (w/w) calicheamicin derivative, 37.5 mM sodium decanoate, 9% (v/v) ethanol, 50 mM HEPBS (N-(2-Hydroxyethyl)piperazine-N′-(4-butanesulfonic acid)), pH 8.5, 32° C., 1 hour. Hydrophobic interaction chromatography (HIC) may be performed using a butyl sepharose FF resin, 0.65 M potassium phosphate loading buffer, 0.49 M potassium phosphate wash buffer, and 4 mM potassium phosphate elution buffer. Buffer exchange may be accomplished by size exclusion chromatography, ultrafiltration/diafiltration, or other suitable means.

As part of the drug-carrier conjugates, mentioned above, N-acetyl gamma dimethyl hydrazide calicheamicin can be conjugated to monoclonal antibodies that specifically bind to the CD22 receptor, the 5T4 receptor and the Lewis-Y antigen, all expressed on cancer cells.

As used herein, the term “reducing agent” refers to a substance that achieves reduction of S—S disulfide bridges. Reduction of the ‘S—S’ disulfide bridges is a chemical reaction whereby the disulfides are reduced to a thiol (—SH). A reducing agent is used to break the disulfide bonds of proteins or other molecules, or as in this invention, a disulfide bond within the immunoconjugate. A reducing agent also maintains the SH group in a reduced state. In some embodiments the reducing agent of the present invention is a thiol reducting agent. Compounds able to reduce disulfide bridges are represented by, but not limited to: dithiothreitol (DTT), dithioerythritol (DTE), mercaptans (e.g., 2-mercaptoethanol), thiocarbamates, Tris(2-carboxyethyl)phosphine and sodium-dichionite.

As used herein, the term “free radical scavenger” refers to any substance that prevents the cascade of chemical reactions that occurs when a free radical reacts with another molecule in order to gain an electron. The molecule that loses an electron to the free radical is transformed into a free radical, repeating the process until two free radicals react with each other, or the reaction is stopped by a free radical scavenger. In the present invention, the reduction of the disulfide bond within the immunoconjugate will generate free radicals after molecular rearrangement of the drug. The free radical scavenger terminates the free radical chain reaction and can thereby stabilize the reaction. Free radical scavengers are represented by, but not limited to: cysteine, acetylcysteine, thioglycollic acid and salts thereof, thiolactic acid and salts thereof, dithiothreitol, reduced glutathione, thiourea, thioglycerol, methionine, mercaptoethane sulfonic acid, an alcohol such as isopropanol, Tris(2-Carboxyethyl)phosphine, benzoic acid, a carbonate ion, a metal complex such as copper complex, manganese complex, sodium hydrogensulfite, sodium sulfite, sodium metabisulfite, nordihydroguaiaretic acid, propyl gallate, butylhydroxyanisole, dibutylhydroxytoluene, erythorbic acid, sodium erythorbate, ascorbyl palmitate, ascorbyl dipalmitate, ascorbyl stearate, sodium ascorbate, calcium ascorbate, glutathione, and uric acid.

As used herein, reference to “disrupting” the bond between calicheamicin and a carrier can include disruption of a S—S bond located at the terminus of calicheamicin that is bound to the carrier through a hydrolysable bifunctional linker. Disruption can also include hydrolysis of a bond within the bifunctional linker portion of the calicheamicin-carrier conjugate. In one embodiment, hydrolysis causes disruption of the hydrazone or Schiff base (RRC═NR) bond.

The present invention provides a method of assaying a sample for the presence of the calicheamicin portion of a calicheamicin-carrier conjugate after a bond linking the calicheamicin to the carrier is disrupted. In one embodiment, the carrier is an antibody covalently bound to the calicheamicin. In one embodiment, the calicheamicin is covalently bound to the antibody by means of an intervening hydrolysable linker. In one embodiment, the linker comprises a (butanoic acid ester).

Linkage of calicheamicin to an antibody by means of a (butanoic acid ester) is described, for example, in U.S. Patent Publication Number 2004019900. Alternative calicheamicin-carrier methods are described in U.S. Pat. Nos. 5,770,710, 5,714,586 and 5,712,374.

In some embodiments, the bond between the carrier and the calicheamicin that is disrupted is in the bifunctional hydrolyzable linker between the calicheamicin and the drug.

In other embodiments, the bond between the carrier and the calicheamicin that is disrupted is between the two sulfur atoms present in the calicheamicin derivative that link the calicheamicin to the bifunctional linker. Upon disulfide bond reduction, the N-acetyl gamma dimethyl hydrazide calicheamicin undergoes molecular rearrangement to form a 1,4-diylradical generated during rearrangement of the enediyne moiety on N-acetyl gamma dimethyl hydrazide calicheamicin. Isopropyl alcohol was used to donate two hydrogen atoms to form N-acetyl epsilon calicheamicin thus acting as a free radical scavenger.

The present invention is further illustrated and supported by the following examples. However, these examples should in no way be considered to further limit the scope of the invention. To the contrary, one having ordinary skill in the art would readily understand that there are other embodiments, modifications, and equivalents of the present invention without departing from the spirit of the present invention and/or the scope of the appended claims.

### EXAMPLES

### General Procedures for Disruption of the Disulfide Bond between Calicheamicin and Antibody and Detection of Calicheamicin

The methods in general utilized: (1) disulfide bond cleavage of a drug-carrier conjugate, calicheamicin and gamma calicheamicin internal standard (IS), (2) formation of N-acetyl epsilon calicheamicin (from drug-carrier conjugates) and epsilon calicheamicin (from gamma calicheamicin IS) after molecular rearrangement, (3) automated liquid-liquid extraction. The extraction procedure includes a) extracting the N-acetyl epsilon calicheamicin and epsilon calicheamicin from the biological or non-biological matrix into organic solvent, such as methyl tertiary-butyl ether, b) evaporating the organic solvent to dryness under a nitrogen stream at approximately 37° C., c) reconstituting the samples into reconstitution solution, which is usually the mixture of HPLC mobile phases. and (4) HPLC separation with mass spectrometry detection.

### Optimization of DTT Concentration

Dithiotreitol (DTT) functions as the disulfide bond reduction agent and hydrogen donor to the free radicals generated in the process of molecular rearrangement of N-acetyl gamma dimethyl hydrazide calicheamicin to 1,4-diylradical. The concentration of DTT was increased to the point before the precipitation of serum protein would occur in the assay. Any precipitation would increase the reaction yield variation. The optimized DTT concentration was shown to be adding 0.1 ml of 0.047 mg/ml DDT in water to 0.05 ml of animal serum. This value is approximately 400 times the calicheamicin ULOQ (1000 ng/ml) in terms of molecular concentrations. At a concentration of 0.47 mg/ml and the volume of 0.1 ml added to 0.05 ml of animal serum no protein precipitation was observed.

### Sample Preparation

A stock solution of approximately 60 μg calicheamicin/mL in water was prepared by dissolving a calicheamicin-antibody conjugate into water (the specific volume of water needed depends on the weight of the conjugate in the vial and the loading capacity (μg of calicheamicin per mg of protein)). The calibration standards and quality control samples in animal serum were fortified using the stock solution and its dilutions. Fifty microliters of each calibration standard, QC or study sample was used for the analysis. Serum samples from rat, monkey or marmoset were spiked with 50 μL IS solution (100 ng/mL in 50% water/50% IPA) and then DTT (dithiothreitol) was added to all samples. Samples sat at room temperature for approximately 1 hour and then liquid-liquid extraction was performed using the Tomtec Quadra 96 liquid handler to add 0.8 ml of methyl t-b ether. The Tomtec Quadra 96 is a liquid handler that is used to transfer liquid automatically by programming. After vortexing, centrifugation and decanting, the samples were evaporated to dryness under a nitrogen stream at approximately 37° C. and then reconstituted into 150 μL of the reconstitution solution (0.1% formic acid in 50% water/50% acetonitrile). The reconstituted samples sat at approximately 4° C. for 40-72 hours prior to LC/MS/MS analysis. It has been discovered that allowing the samples to sit for 4° C. for a time period improves the precision and accuracy of the quantitation of the disrupted calicheamicin.

### LC/MS/MS Conditions

HPLC separation was performed using 20 microliters of sample injected onto a YMC Pro, C₁₈ (2.0×50mm, 3 micron) column with a flow rate of 0.4 ml/minute with a run time of 4.6 minutes. Compounds were then eluted at ambient temperature using mobile phases of 0.1% formic acid in water and 0.1% formic acid in acetonitrile. Mass spectrometry was then performed using an Applied Biosystems/MDS Sciex API 4000 in positive ion mode. The linear calibration curve was constructed using the nominal calicheamicin concentrations in the calibration standards and their respective instrument response ratio (calicheamicin peak area/IS peak area). The calibration curve was then used to calculate calicheamicin concentration in each sample using the instrument response ratio.

### Calibration Curves

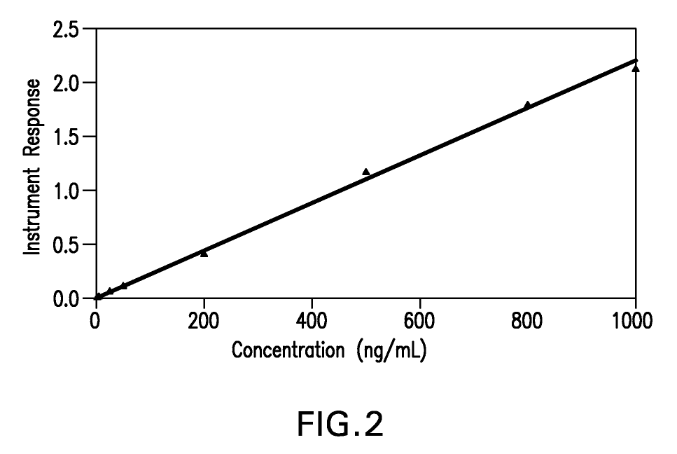

Antibody-drug conjugates CME-548 (55.8 μg calicheamicin/mg of protein, 0.98 mg protein per vial) or CMC-544 (66 μg calicheamicin/mg of protein, 0.96 mg protein per vial), were used to prepare calicheamicin stock solutions. These stock solutions were then used to generate a calibration curve for known concentrations of calicheamicin added to rat, monkey or marmoset serum. FIG. 2 shows a representative calibration standard curve for calicheamicin quantitation with mass spectrophotometry. Calibration standards are serial dilutions used to quantify the amount of calicheamicin detected with analytical mass spectrophotometry.

CMC-544 is a calicheamicin-monoclonal antibody drug conjugate described in U.S. patent application Publication No. 2004-082764A1 and U.S. patent application Publication No. 20060088522. The monoclonal antibody portion of the conjugate is specific for the CD22 receptor.

CME-548 is a calicheamicin-monoclonal antibody drug conjugate described in U.S. patent application Publication No. US2006008522. The monoclonal antibody portion of the conjugate is specific for the 5T4 receptor.

### Example 1

### Quantitation of Calicheamicin in Sera Following Disruption of Disulfide Bond

Various concentrations of calicheamicin-antibody conjugate were added to sera from different species. The disulfide bond was disrupted and total calicheamicin was determined as described above.

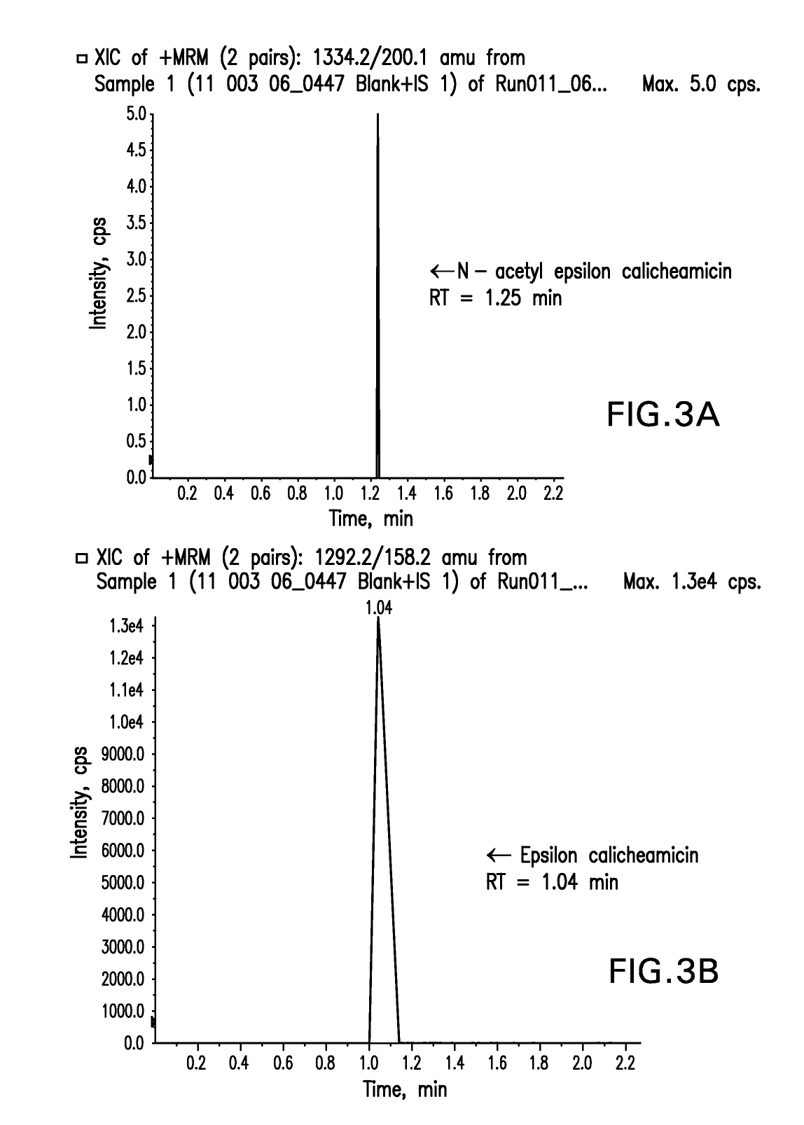

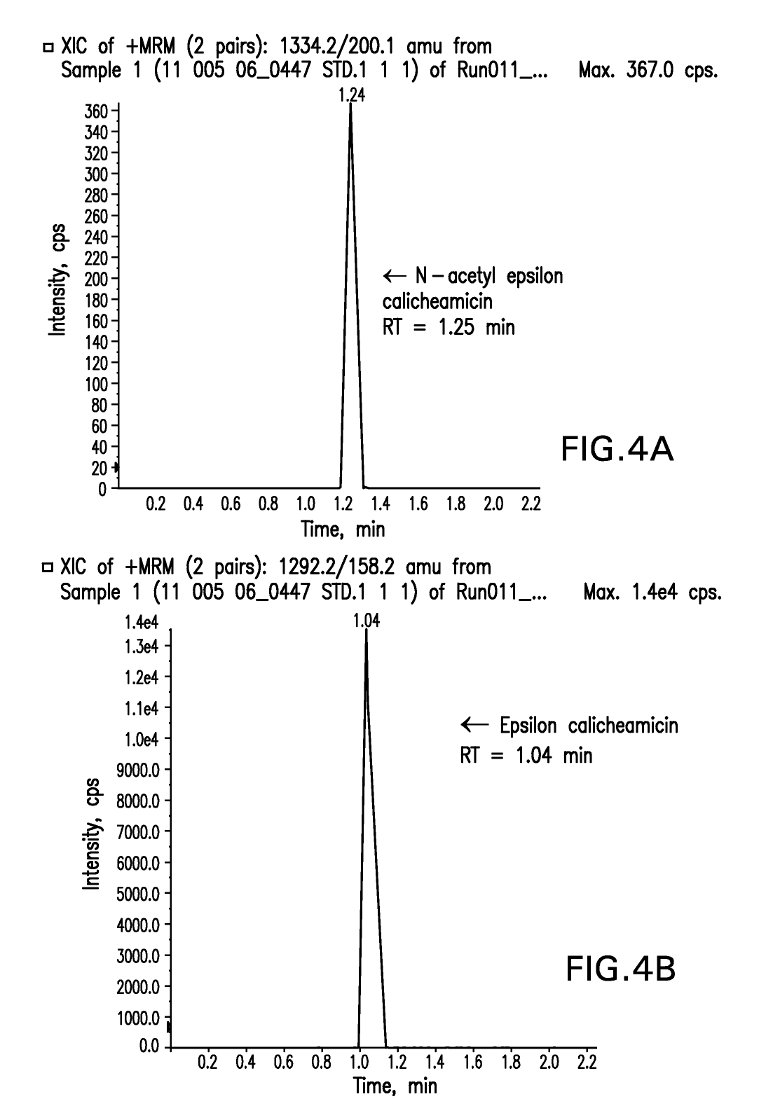

To determine the amount of calicheamicin present in a sample, in one assay the calibration standards were analyzed over time and the concentrations of calicheamicin in the calibration standards were back calculated. Tables 1, 2 and 3 show the back calculation of calibration standards, along with the average, standard deviation and CV (coefficient of variance) in monkey (CMC-544), and rat (CME-548, CMC-544) sera. Readouts for mass spectrometry are observed and recorded in counts per minute. Examples for typical readouts of mass spectrophotometry data used to detect and quantify the amount of calicheamicin are shown in FIGS. 3A and B and 4A and B. FIGS. 3A and 3B show representative chromatograms of control rat serum with IS. FIGS. 4A and 4B show representative chromatograms of CME-548 LLOQ calibration standard (2.00 ng calicheamicin/mL) in rat serum.

TABLE 1
ANALYTICAL PERFORMANCE OF CMC-544 ASSAY IN MONKEY SERUM:
BACK-CALCULATED CONCENTRATIONS OF CALIBRATION STANDARDS

| | | Nominal Concentrations (ng/mL) | | | | | | | |
|---|---|---|---|---|---|---|---|---|---|
| Assay | Analytical | 2.00 | 5.00 | 25.0 | 50.0 | 200 | 500 | 800 | 1000 |
| Date | Run Number | Measured Concentrations (ng/mL)^{a} | | | | | | | |
| 19-May-2006 | 5 | 1.99 | 6.43^{b} | 25.8 | 51.8 | 215 | 489 | 749 | 947 |
| 21-May-2006 | 6 | 1.91 | 5.50 | 43.9^{b} | 53.4 | 222 | 492 | 743 | 853 |
| 30-May-2006 | 8 | 2.01 | 6.16^{b} | 21.7 | 55.4 | 183 | 477 | 839 | 1098 |
| 31-May-2006 | 9 | 2.04 | 4.71 | 25.6 | 50.6 | 206 | 519 | 774 | 964 |
| | Mean | 1.99 | 5.11 | 24.4 | 52.8 | 207 | 494 | 776 | 966 |
| | S.D. | 0.0556 | NA | 2.31 | 2.08 | 17.0 | 17.7 | 43.9 | 101 |
| | % CV | 2.8 | NA | 9.5 | 3.9 | 8.2 | 3.6 | 5.7 | 10.5 |
| | % Bias | −0.6 | 2.1 | −2.5 | 5.6 | 3.3 | −1.2 | −3.0 | −3.5 |
| | N | 4 | 2 | 3 | 4 | 4 | 4 | 4 | 4 |

^{a}A linear regression method was used with 1/concentration² as the weighting factor.
^{b}Calibrator deactivated from the standard curve regression (bias > 15%).
NA: not applicable

TABLE 2
ANALYTICAL PERFORMANCE OF CME-548 ASSAY IN RAT SERUM:
BACK-CALCULATED CONCENTRATIONS OF CALIBRATION STANDARDS

| | | Calicheamicin Nominal Concentrations (ng/mL) | | | | | | | |
|---|---|---|---|---|---|---|---|---|---|
| Assay | Analytical | 2.00 | 5.00 | 25.0 | 50.0 | 200 | 500 | 800 | 1000 |
| Date | Run Number | Analyte Measured Concentrations (ng/mL)^{a} | | | | | | | |
| 21-May- 2006 | 11 | 2.04 | 4.67 | 25.9 | 52.7 | 199 | 508 | 794 | 951 |
| 23-May-2006 | 12 | 1.92 | 5.48 | 25.5 | 49.2 | 193 | 496 | 803 | 980 |
| 24-May-2006 | 13 | 2.06 | 4.63 | 24.4 | 50.0 | 208 | 490 | 818 | 1023 |
| 28-May-2006 | 15 | 2.04 | 4.70 | 25.2 | 50.5 | 212 | 504 | 789 | 966 |
| 07-Jun-2006 | 16 | 2.01 | 4.80 | 26.8 | 54.4 | 211 | 483 | 735 | 938 |
| 16-Jun-2006 | 17 | 1.97 | 5.04 | 28.2 | 56.0 | 196 | 467 | 774 | 881 |
| | Mean | 2.01 | 4.89 | 26 | 52.1 | 203 | 491 | 786 | 957 |
| | SD | 0.0528 | 0.326 | 1.34 | 2.69 | 8.18 | 15 | 28.7 | 47.2 |
| | % CV | 2.6 | 6.7 | 5.2 | 5.2 | 4.0 | 3.1 | 3.7 | 4.9 |
| | % Bias | 0.5 | −2.2 | 4.0 | 4.2 | 1.5 | −1.8 | −1.8 | −4.3 |
| | n | 6 | 6 | 6 | 6 | 6 | 6 | 6 | 6 |

^{a}A linear regression method was used with 1/concentration² as the weighting factor.

TABLE 3
ANALYTICAL PERFORMANCE OF CMC-544 ASSAY IN
RAT SERUM: CALIBRATION CURVE PARAMETERS

| Run Date | Curve Number^{a} | Slope | Intercept | r² |
|---|---|---|---|---|
| 23-Jun-06 | 4 | 0.00863 | 0.002340 | 0.9954 |
| 26-Jun-06 | 5 | 0.01100 | −0.003680 | 0.9965 |
| 28-Jun-06 | 6 | 0.00618 | 0.000740 | 0.9980 |
| 28-Jun-06 | 7 | 0.00580 | 0.002470 | 0.9951 |
| 31-Jul-06 | 8 | 0.00479 | −0.000473 | 0.9992 |
| | Mean | 0.00728 | 0.000279 | 0.9968 |
| | S.D. | 0.00251 | 0.00252 | 0.0017 |
| | n | 5 | 5 | 5 |

^{a}A linear regression method was used with 1/concentration² as the weighting factor.

### Example 2

### Validation of Detection of Total Calicheamicin in Sera following Disruption of Calicheamicin-Antibody Conjugate

Validation studies were performed with CMC-544 in rat (Table 4) and monkey (Table 5) sera and for CME-548 in rat serum (Table 6). Intra day results represent three different runs of 5 samples each performed three times on the same day. Inter day results represent a summary of 5 samples prepared each day for three consecutive days.

Table 4 shows the relative standard deviation and % accuracy for Intra and Inter day analyses of low, medium and high concentrations of calicheamicin derived from CMC-544 in rat sera.

Table 5 shows the relative standard deviation and % accuracy for Intra and Inter day analyses of low, medium and high concentrations of calicheamicin derived from CMC-544 in monkey sera.

Table 6 shows the relative standard deviation and % accuracy for Intra and Inter day analyses of low, medium and high concentrations of calicheamicin-derived from CME-548 in rat sera.

Results for Tables 4, 5 and 6 demonstrate that the assay for total calicheamicin is repeatable with accurate and precise determination of calicheamicin concentrations.

TABLE 4
ANALYTICAL PERFORMANCE OF CMC-544 ASSAY IN
RAT SERUM: INTRA-DAY AND INTER-DAY PRECISION
AND BIAS

| | Nominal Concentrations | | |
|---|---|---|---|
| | Low | Mid | High |
| | (6.00 ng/mL) | (125 ng/mL) | (750 ng/mL) |
| Run Number | Measured Concentrations (ng/mL) | | |
| 4 | 6.42 | 120 | 742 |
| | 6.47 | 123 | 720 |
| | 10.7^{a} | 118 | 758 |
| | 6.58 | 126 | 669 |
| | 6.50 | 119 | 665 |
| Mean | 6.49 | 121 | 711 |
| S.D. | 0.0690 | 3.36 | 42.4 |
| % CV | 1.1 | 2.8 | 6.0 |
| % Bias | 8.2 | −2.9 | −5.2 |
| n | 4 | 5 | 5 |
| 6 | 6.69 | 139 | 756 |
| | 5.74 | 119 | 662 |
| | 6.42 | 140 | 698 |
| | 5.82 | 130 | 662 |
| | 6.12 | 124 | 665 |
| Mean | 6.16 | 130 | 688 |
| S.D. | 0.401 | 9.45 | 40.6 |
| % CV | 6.5 | 7.2 | 5.9 |
| % Bias | 2.6 | 4.3 | −8.2 |
| n | 5 | 5 | 5 |
| 7 | 6.29 | 111 | 657 |
| | 7.92 | 133 | 592 |
| | 7.12 | 121 | 724 |
| | 8.14 | 129 | 589 |
| | 6.10 | 114 | 732 |
| Mean | 7.11 | 121 | 659 |
| S.D. | 0.922 | 9.25 | 68.9 |
| % CV | 13.0 | 7.6 | 10.5 |
| % Bias | 18.6 | −2.8 | −12.1 |
| n | 5 | 5 | 5 |
| Overall Mean | 6.59 | 124 | 686 |
| Overall S.D. | 0.702 | 8.50 | 53.2 |
| Overall % CV | 10.6 | 6.8 | 7.8 |
| Overall % Bias | 9.9 | −0.5 | −8.5 |
| n | 14 | 15 | 15 |

TABLE 5
ANALYTICAL PERFORMANCE OF CMC-544 ASSAY IN
MONKEY SERUM: INTRA-DAY AND INTER-DAY
PRECISION AND BIAS

| | Nominal Concentrations | | |
|---|---|---|---|
| | Low | Mid | High |
| | (6.00 ng/mL) | (125 ng/mL) | (750 ng/mL) |
| Run Number | Measured Concentrations (ng/mL) | | |
| 5 | 5.89 | 126 | 682 |
| | 6.62 | 133 | 707 |
| | 6.28 | 123 | 666 |
| | 6.13 | 131 | 749 |
| | 5.64 | 125 | 697 |
| Mean | 6.11 | 128 | 700 |
| S.D. | 0.374 | 4.22 | 31.4 |
| % CV | 6.1 | 3.3 | 4.5 |
| % Bias | 1.9 | 2.1 | −6.6 |
| n | 5 | 5 | 5 |
| 6 | 6.86 | 128 | 709 |
| | 6.64 | 132 | 694 |
| | 6.47 | 139 | 686 |
| | 5.90 | 126 | 730 |
| | 6.53 | 132 | 705 |
| Mean | 6.48 | 132 | 705 |
| S.D. | 0.356 | 5.00 | 16.6 |
| % CV | 5.5 | 3.8 | 2.4 |
| % Bias | 8.0 | 5.2 | −6.0 |
| N | 5 | 5 | 5 |
| 9 | 5.70 | 124 | 695 |
| | 5.07 | 130 | 716 |
| | 5.76 | 129 | 709 |
| | 5.82 | 132 | 681 |
| | 5.81 | 113 | 690 |
| Mean | 5.63 | 125 | 698 |
| S.D. | 0.316 | 7.54 | 14.1 |
| % CV | 5.6 | 6.0 | 2.0 |
| % Bias | −6.1 | 0.4 | −6.9 |
| N | 5 | 5 | 5 |
| Overall Mean | 6.08 | 128 | 701 |
| Overall S.D. | 0.483 | 5.93 | 20.6 |
| Overall % CV | 8.0 | 4.6 | 2.9 |
| Overall % Bias | 1.3 | 2.6 | −6.5 |
| N | 15 | 15 | 15 |

TABLE 6
ANALYTICAL PERFORMANCE OF CME-548 ASSAY IN
RAT SERUM: INTRA-DAY AND INTER-DAY PRECISION
AND BIAS

| | Calicheamicin Nominal Concentrations | | |
|---|---|---|---|
| | Low | Mid | High |
| | (6.00 ng/mL) | (125 ng/mL) | (750 ng/mL) |
| Run Number | Analyte Measured Concentrations (ng/mL) | | |
| 11 | 6.94 | 132 | 760 |
| | 6.24 | 130 | 836 |
| | 6.24 | 133 | 803 |
| | 6.97 | 129 | 811 |
| | 6.03 | 125 | 816 |
| Mean | 6.48 | 130 | 805 |
| SD | 0.439 | 3.16 | 28.0 |
| % CV | 6.8 | 2.4 | 3.5 |
| % Bias | 8.0 | 3.9 | 7.4 |
| n | 5 | 5 | 5 |
| 12 | 6.94 | 134 | 721 |
| | 6.95 | 138 | 711 |
| | 7.36 | 137 | 707 |
| | 6.58 | 140 | 708 |
| | 7.20 | 138 | 755 |
| Mean | 7.01 | 137 | 720 |
| SD | 0.299 | 2.28 | 19.9 |
| % CV | 4.3 | 1.7 | 2.8 |
| % Bias | 16.8 | 9.9 | −3.9 |
| n | 5 | 5 | 5 |
| 13 | 6.25 | 131 | 847 |
| | 7.31 | 131 | 845 |
| | 6.38 | 134 | 827 |
| | 7.05 | 133 | 853 |
| | 6.58 | 130 | 834 |
| Mean | 6.71 | 132 | 841 |
| SD | 0.449 | 1.51 | 10.4 |
| % CV | 6.7 | 1.1 | 1.2 |
| % Bias | 11.9 | 5.5 | 12.2 |
| N | 5 | 5 | 5 |
| Overall Mean | 6.73 | 133 | 789 |
| Overall S.D. | 0.432 | 4.00 | 55.9 |
| Overall % CV | 6.4 | 3.0 | 7.1 |
| Overall % Bias | 12.2 | 6.4 | 5.2 |
| N | 15 | 15 | 15 |

### Example 3

### Determination of Total Calicheamicin following Administration of Antibody-Calicheamicin Conjugate to an Animal Subject

Calicheamicin-drug carrier conjugate CME-548 was administered to animals by intravenous injection to male marmosets at dosages of 0 (vehicle-control), 7 (low), 25 (mid) and 75 (high) μg/kg of calicheamicin equivalents. Serum samples were collected at various time intervals ranging on day 8 and total calicheamicin (measured as N-acetyl epsilon calicheamicin) concentrations in serum samples were determined.

A stock solution of CME-548 (60 μg calicheamicin/mL water) was prepared. Calibration standards and quality control samples were fortified using the stock solution and its dilutions. Fifty microliters of each calibration standard, quality control sample or animal samples were used for analysis. Samples were spiked with IS and then DTT was added to all samples. Samples were incubated at room temperature for approximately 1 hour and then extracted with methyl t-b ether using the Tomtec Quadra 96 liquid handler. After vortexing, centrifugation and decanting, the samples were evaporated to dryness under a nitrogen stream at approximately 37° C., reconstituted into the reconstitution solution (0.1% formic acid in 50% water/50% acetonitrile), and allowed to sit at 4° C. for 40-72 hours prior to LC/MS/MS analysis.

### LC/MS Conditions

HPLC separation was then performed using 20 microliters of sample injected onto a YMC Pro, C₁₈ (2.0×50 mm, 3 micron) column with a flow rate of 0.4 ml/minute with a run time of 4.6 minutes. Compounds were then eluted at ambient temperature using mobile phases of 0.1% formic acid in water and 0.1% formic acid in acetonitrile. Mass spectrometry was then performed using an Applied Biosystems/MDS Sciex API 4000 in positive ion mode. Readouts for mass spectrometry were observed in counts per minute. The determined total calicheamicin concentrations in marmoset serum are shown in table 7.

TABLE 7
SERUM CONCENTRATIONS (NG/ML) OF TOTAL
CALICHEAMICIN IN MALE MARMOSETS FOLLOWING
INTRAVENOUS (BOLUS) DOSE OF CME-548 AT 0
(VEHICLE CONTROL), 7, 25, AND 75 MG/KG
(CALICHEAMICIN EQUIVALENTS): DAY 8 DATA

| Dosage (μg/kg) | SAN | Concentration (ng/mL) |
|---|---|---|
| 0 | 1 | <2.00 |
| | 2 | <2.00 |
| | 3 | <2.00 |
| | 4 | <2.00 |
| | Mean | 0 |
| | S.D. | 0 |
| | n | 4 |
| 7 | 5 | 15.5 |
| | 6 | 20.2 |
| | 7 | 16.4 |
| | Mean | 17.4 |
| | S.D. | 2.49 |
| | n | 3 |
| 25 | 8 | 80.5 |
| | 9 | 65.8 |
| | 10 | 60.1 |
| | Mean | 68.8 |
| | S.D. | 10.5 |
| | n | 3 |
| 75 | 11 | 303 |
| | 12 | 194 |
| | 13 | 177 |
| | Mean | 225 |
| | S.D. | 68.4 |
| | n | 3 |

SAN. Study Animal Number.

### Example 4

### Hydrolysis of the Linker Between Calicheamicin and the Antibody

Calicheamicin is hydrolyzed from the antibody conjugate at the linker between about a pH of 3.0 and pH 4.0 for 1-24 hours at a temperature from 20 to 50° C., preferably 37° C. The analyte is extracted from animal serum by liquid-liquid extraction using an organic solvent such as ethyl acetate, ethyl ether or MTBE. Alternatively, the analyte is extracted by protein precipitation using agents such as acetonitrile, acetone or methanol, by solid-phase extraction using reverse phase or mix mode solid phase extraction cartridges. (Either of the above alternatives, or mix mode solid phase can be used). The extracted samples are evaporated and reconstituted prior to being analyzed using an LC/MS/MS system.

1. A method of detecting the presence of the calicheamicin component of a calicheamicin-carrier conjugate in a fluid sample, wherein the calicheamicin is covalently bound to the carrier, the method comprising the following steps:
(a) disrupting a covalent bond between calicheamicin and the carrier component; and
(b) detecting the calicheamicin portion that is released from the calicheamicin-carrier conjugate.

2. The method of claim 1, wherein the calicheamicin released from the calicheamicin-carrier conjugate is detected by mass spectrometry.

3. The method of claim 1, wherein the covalent bond between calicheamicin and the carrier is disrupted by adding a reducing agent and the disrupted bond is the disulfide bond linking the calicheamicin to the carrier.

4. The method of claim 3, wherein the reducing agent comprises at least one of dithiothreitol (DTT), dithioerythritol (DTE), a mercaptan, glutathione, thiocarbamates, tris(2-carboxyethyl)phosphine (TCEP) and sodium-dichionite.

5. The method of claim 3 wherein the reducing agent is a thiol reducing agent.

6. The method of claim 5, wherein the thiol reducing agent is dithiothreitol (DTT).

7. The method of claim 3, which further comprises the step of adding a free radical scavenger to the sample.

8. The method of claim 7, wherein the free radical scavenger is selected from the group consisting of: an alcohol, a thiol derivative, Tris(2-Carboxyethyl)phosphine; benzoic acid; a carbonate ion; a copper complex, a manganese complex; sodium hydrogensulfite; sodium sulfite; sodium metabisulfite; nordihydroguaiaretic acid; propyl gallate; butylhydroxyanisole, dibutylhydroxytoluene; erythorbic acid; sodium erythorbate, ascorbyl palmitate, ascorbyl dipalmitate; ascorbyl stearate; sodium ascorbate; calcium ascorbate; glutathione; and uric acid.

9. The method of claim 8, wherein the free radical scavenger is an alcohol comprising isopropyl alcohol.

10. The method of claim 1 wherein the calicheamicin detected is N-acetyl epsilon calicheamicin.

11. The method of claim 1 wherein the disrupting step in (a) is incubated for a time period between about 0.5 hour and about 3 hours.

12. The method of claim 11, which further comprises an incubation step prior to detecting the calicheamicin.

13. The method of claim 12, wherein the incubation step is maintained for a time period between about 24 hours and about 96 hours.

14. The method of claim 13, wherein the incubation step is maintained for a time period of about 48 hours.

15. The method of claim 12, wherein the incubation step is at a temperature between about 0° C. and 100° C.

16. The method of claim 15, wherein the incubation step is incubated at a temperature between about 0° C. and 25° C.

17. The method of claim 15, wherein the incubation step is incubated at a temperature of about 4° C.

18. The method of claim 1, wherein the disrupted bond is in the linker portion of the calicheamicin-carrier conjugate and the linker is hydrolyzed.

19. The method of claim 18 wherein a bond in the linker is disrupted by incubation of the sample in a pH between about 2.0 and 4.0 for a time period between about 0.5 hour and about 24 hours.

20. The method of a claim 2, wherein detection by mass spectrometry is selected from the group of assays consisting of: Surface Enhanced Laser Desorption Ionization (SELDI); Matrix-Assisted Laser Desorption/Ionization quadropole time-of-flight (MALDI-TOF); multiple sequential mass spectrometry (MS/MS), sequential time-of-flight (TOF-TOF), electrospray ionization quadropole time-of-flight (ESI-O-TOF) and ION-TRAP.

21. The method of claim 20, wherein the mass spectrometry is multiple sequential spectrometry and comprises liquid chromatography (LC/MS/MS).

22. The method of claim 1, wherein the carrier component of the calicheamicin-carrier conjugate is selected from the group consisting of: mono- and polyclonal antibodies and their chemically or genetically manipulated counterparts; their antigen-recognizing fragments and their chemically or genetically manipulated counterparts; small modular immunopharmaceuticals (SMIPs) and their chemically or genetically manipulated counterparts; nanobodies and their chemically or genetically manipulated counterparts; soluble receptors and their chemically or genetically manipulated counterparts; and growth factors and their chemically or genetically manipulated counterparts; aptamers; liposomes; non-glycosylated proteins; and nanoparticles.

23. The method of claim 22, wherein the carrier is one that can specifically bind to an antigen expressed on the surface of cancer cells.

24. The method of claim 23, wherein the antigen expressed on the cancer cells is selected from the group consisting of: 5T4; CD19; CD20; CD22; CD33; CD40; EphA2; Lewis Y; HER-2; type I Fc receptor for immunoglobulin G (Fc gamma R1); CD52; epidermal growth factor receptor (EGFR); vascular endothelial growth factor (VEGF); DNA/histone complex; carcinoembryonic antigen (CEA); CD47; CD105 (endoglin); folate R; CSPG4 (melanoma-associated antigen); MUC-1; prostate specific membrane antigen (PSMA); VEGFR2 (vascular endothelial growth factor receptor 2 or kinase insert domain-containing receptor, KDR); epithelial cell adhesion molecule (Ep-CAM); fibroblast activation protein (FAP); Trail receptor-1 (DR4); progesterone receptor; oncofetal antigen CA 19.9; and fibrin.

25. The method of claim 22 wherein the carrier component of the calicheamicin-carrier conjugate is a monoclonal antibody.

26. A kit for determining the concentration of calicheamicin present in a sample comprising: instructions for disrupting a disulfide bond between calicheamicin and an antibody; a free radical scavenger; and a reducing agent.

27. The kit of claim 26 further comprising N-acetyl gamma dimethyl hydrazide.

28. The kit of claim 27 wherein the reducing agent comprises DTT.

29. The kit of claim 26 wherein the free radical scavenger comprises at least one of the following: an alcohol; a thiol derivative; Tris(2-Carboxyethyl)phosphine; benzoic acid; a carbonate ion; copper complex, manganese complex; sodium hydrogensulfite; sodium sulfite; sodium metabisulfite; nordihydroguaiaretic acid; propyl gallate; butylhydroxyanisole; dibutylhydroxytoluene; erythorbic acid; sodium erythorbate; ascorbyl palmitate; ascorbyl dipalmitate; ascorbyl stearate; sodium ascorbate; calcium ascorbate; glutathione; and uric acid.
